(19) United States
(12) Patent Application Publication
(10) Pub. No.: US 2024/0242821 A1
(43) Pub. Date: Jul. 18, 2024

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Yoshiteru WATANABE, Nasushiobara (JP); Shinya OZAWA, Saitama (JP); Takeshi ISHIMOTO, Arakawa (JP)

(73) Assignee: CANON MEDICAL SYSTEMS CORPORATION, Otawara-shi (JP)

(21) Appl. No.: 18/411,244

(22) Filed: Jan. 12, 2024

(30) Foreign Application Priority Data
Jan. 13, 2023 (JP) 2023-003774

(51) Int. Cl. G16H 40/20; A61B 5/00; G08B 25/00; G16H 30/40

(52) U.S. Cl. CPC G16H 40/20; A61B 5/7465; G08B 25/005; G16H 30/40

(57) ABSTRACT

A medical information processing device of an embodiment includes processing circuitry. The processing circuitry is configured to acquire a call request for calling a user to a medical image diagnostic device, calculate a notification priority for each of a plurality of users on the basis of at least one of usage statuses of a plurality of terminals managed by the plurality of users or details of work of the plurality of users, and transmit notification information for causing the plurality of terminals to output a call notification according to the notification priority to the plurality of terminals.

### CROSS-REFERENCE TO RELATED APPLICATION

The present application claims priority based on Japanese Patent Application No. 2023-003774 filed Jan. 13, 2023, the content of which is incorporated herein by reference.

### FIELD

Embodiments disclosed in this specification and drawings relate to a medical information processing device, a medical information processing method, and a storage medium.

### BACKGROUND

In recent years, there has been a technology by which a technician or the like can remotely operate a medical image diagnostic device (hereinafter, as a modality) using a mobile terminal (tablet terminal) at a location away from the modality. Conventionally, one tablet terminal is associated with one modality, and a modality can only be operated using the associated tablet terminal. On the contrary, a technology by which modalities can be operated using a plurality of tablet terminals has been developed. By using this technology, a plurality of technicians or the like can carry tablet terminals and operate a plurality of modalities.

Under such circumstances, when work is being performed using a modality with no technician nearby, it may be necessary for a technician or the like to respond to the modality. In this case, the modality can send a notification requesting a response to each of a plurality of mobile terminals. However, even if a plurality of technicians can respond, a technician who is most easily able to respond may not respond appropriately and a technician who has difficulty in responding may be required to respond.

### BRIEF DESCRIPTION OF THE DRAWINGS

FIG. 1 is a block diagram showing an example of a configuration of an in- hospital system 1 of a first embodiment.

FIG. 2 is a block diagram showing an example of a configuration of a tablet terminal 50 of the first embodiment.

FIG. 3 is a diagram showing an example of a notification method list 59 of the first embodiment.

FIG. 4 is a block diagram showing an example of a configuration of a medical information processing device 100 of the first embodiment.

FIG. 5 is a diagram showing an example of a first priority order list 151 of the first embodiment.

FIG. 6 is a diagram showing an example of a second priority order list 152 of the first embodiment.

FIG. 7 is a flowchart showing an example of processing in the medical information processing device 100 of the first embodiment.

FIG. 8 is a flowchart showing an example of processing in the medical information processing device 100 of the first embodiment.

FIG. 9 is a flowchart showing an example of processing in a medical information processing device 100 of a modified example.

FIG. 10 is a flowchart showing an example of processing in a medical information processing device 100 of a second embodiment.

FIG. 11 is a block diagram showing an example of a configuration of an in- hospital system 1 of a third embodiment.

FIG. 12 is a flowchart showing an example of processing in a medical information processing device 100 of a fourth embodiment.

### DETAILED DESCRIPTION

Hereinafter, a medical information processing device, a medical information processing method, and a storage medium according to embodiments will be described with reference to the drawings.

A medical information processing device of an embodiment includes processing circuitry. The processing circuitry is configured to acquire a call request for calling a user to a medical image diagnostic device, calculate a notification priority for each of a plurality of users on the basis of at least one of usage statuses of a plurality of terminals managed by the plurality of users or details of work of the plurality of users, and transmit notification information for causing the plurality of terminals to output a call notification according to the notification priority to the plurality of terminals.

### (FIRST EMBODIMENT)

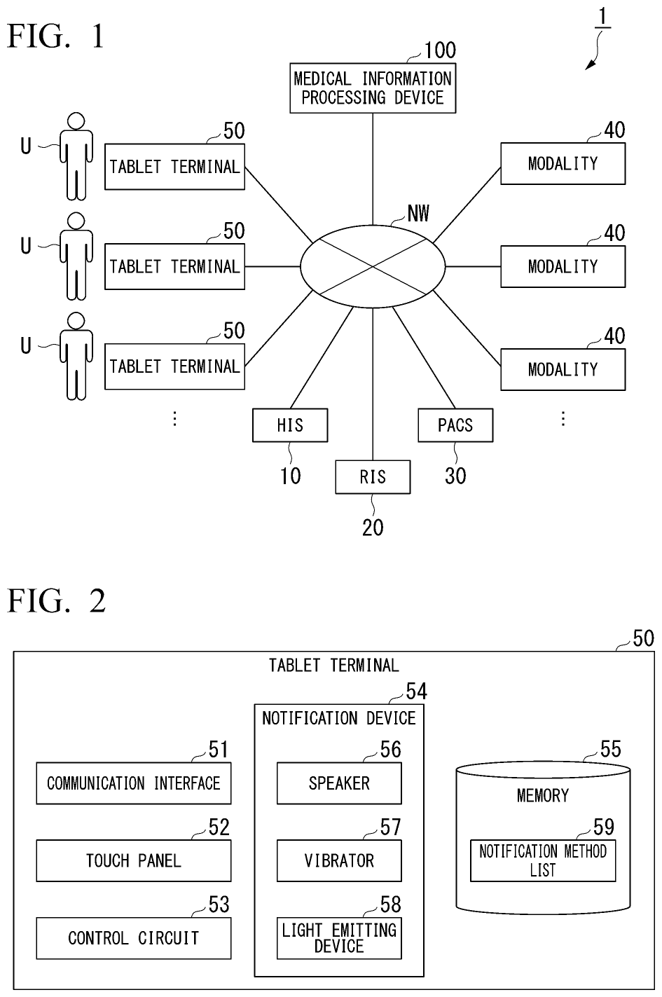

FIG. 1 is a block diagram showing an example of a configuration of an in- hospital system 1 of a first embodiment. The in-hospital system 1 of the first embodiment includes, for example, a hospital information system (hereinafter, HIS) 10, a radiology information system (hereinafter, RIS) 20, a picture archiving and communication system (PACS) 30, a plurality of medical image diagnostic devices (modalities) 40, a plurality of tablet terminals 50, and a medical information processing device 100.

The HIS 10 is a computer system that supports work within the hospital. Specifically, the HIS 10 has various subsystems. The various subsystems include, for example, an electronic medical records system, a medical accounting system, a medical reservation system, a hospital visit reception system, and an admission/discharge management system.

The HIS 10 includes, for example, a computer such as a server device or a client terminal equipped with a processor such as a central processing unit (CPU), a memory such as a read only memory (ROM) or a random access memory (RAM), a display, an input interface, and a communication interface.

A technician or the like (hereinafter, a user) U inputs or refers to information regarding a patient using the electronic medical record system included in the HIS 10. A user U issues an image examination order to the HIS 10. The HIS 10 transfers order information corresponding to the image examination order to other systems such as the RIS 20.

The HIS 10 manages work schedules of users U and the modalities 40. The HIS 10 determines a user U who will be in charge of image examination and a modality 40 to be used for image examination on the basis of the issued image examination order. The HIS 10 manages schedules of the user U and the modality 40, including the determined details. The HIS 10 transmits the schedules of the user U and the modality 40 to the medical information processing device 100 via a network NW in response to a request from the medical information processing device 100.

The RIS 20 is a computer system that supports work in an image diagnosis department. The RIS 20 performs association of reservation information with examination equipment, management of examination information, and the like in addition to reservation management of image examination orders in association with the

HIS 10. The RIS 20 includes, for example, a computer such as a server device or a client terminal that includes a processor such as a CPU, a memory such as a ROM or a RAM, a display, an input interface, and a communication interface.

The PACS 30 is a computer system that receives medical images transmitted by the modalities 40 and stores the medical images in a database. The PACS 30 transmits (transfers) medical images stored in the database in response to a request from a client. The PACS 30 includes a server computer including a processor such as a CPU, a memory such as a ROM and a RAM, a display, an input interface, and a communication interface. Medical images stored in the PACS 30 are accompanied by information regarding patients to be imaged and imaging as supplementary information. The supplementary information includes information such as patient IDs, examination IDs, and imaging conditions (imaging protocol) in a format that complies with the Digital

Imaging and Communication in Medicine (DICOM) standard, for example. The modality 40 executes image-capturing (imaging) according to imaging conditions (imaging protocol) determined on the basis of an image examination instruction or the like, for example. Examples of the modality 40 include an X-ray computed tomography device, an X-ray diagnostic device, a magnetic resonance imaging device, an ultrasound diagnostic device, a nuclear medicine diagnostic device, and the like.

The modality 40 includes, for example, a pedestal, a bed, and a console. The pedestal is also called a gantry. In the case of an X-ray tomography device which is a device for taking an image of a subject, the pedestal is provided with, for example, a tube that radiates X-rays, an X-ray detector that detects X-rays, and the like. The bed carries a subject. The console controls various devices provided on the pedestal, designates output of equipment provided on the pedestal, and generates images on the basis of information provided by equipment provided on the pedestal. A user U can operate the modality 40 using the console. The user U can operate the modality 40 not only through the console but also through the tablet terminal 50 managed by the user. Medical images (image data) generated by imaging performed by the modality 40 are transmitted to the PACS 30.

The modality 40 performs an examination that involves taking images of a subject. Details of the examination performed by the modality 40 (hereinafter, details of the examination of the modality 40) include, for example, “checkup or examination of mild case,” “non-contrast imaging examination (head),” “non-contrast imaging examination (other than the head),” and “contrast imaging examination.”

The modality 40 includes an input interface for receiving information regarding a subject and information regarding imaging (hereinafter, examination-related information) and a communication interface for communicating with the plurality of tablet terminals 50 via the network NW such as a local area network (LAN). The communication interface includes, for example, a communication interface such as a network interface card (NIC). The network NW may include the Internet, a cellular network, a Wi-Fi network, a wide area network (WAN), and the like instead of or in addition to a LAN. The modality 40 receives examination-related information transmitted by the tablet terminals 50 through the communication interface.

The modality 40 transmits a call request to the medical information processing device 100 in cases in which a treatment by the user U is required during an examination. Cases in which treatment by the user U is required during an examination include, for example, cases in which the modality 40 under examination breaks down or stops and cases in which an abnormality has occurred in a subject.

The configuration of the in-hospital system 1 is not limited to the above. The in-hospital system 1 mayinclude, for example, an image interpretation report creation device and the like. Moreover, some elements of the in-hospital system 1 maybe integrated. For example, the HIS 10 and the RIS 20 maybe integrated into one system.

FIG. 2 is a block diagram showing an example of a configuration of the tablet terminals 50 of the first embodiment. The tablet terminals 50 are distributed to a plurality of users U, respectively. Each user U manages the tablet terminal 50 distributed thereto. The tablet terminal 50 is a mobile terminal carried by each user U.

The number of tablet terminals 50 managed by a user U is one, and one tablet terminal 50 is assigned to one user U. The tablet terminals 50 maybe assigned to a plurality of users U, or one tablet terminal 50 maybe assigned to a plurality of users U. Information on users U to whom the tablet terminals 50 have been distributed is managed by the medical information processing device 100, for example.

The tablet terminal 50 includes, for example, a communication interface 51, a touch panel 52, a control circuit 53, a notification device 54, and a memory 55. The notification device 54 includes, for example, a speaker 56, a vibrator 57, and a light emitting device 58. The communication interface 51 communicates with the medical information processing device 100 and the plurality of modalities 40 via a network NW such as a LAN, for example. The communication interface 51 receives notification information transmitted by the medical information processing device 100, for example.

The touch panel 52 serves as an input interface through which the user U inputs information and an output interface for displaying input information. The touch panel 52 displays, for example, a graphical user interface (GUI) or the like for receiving various input operations from the user U. The GUI includes, for example, one for the user U to remotely operate the modality 40. The touch panel 52 transmits information corresponding to a received input operation to the control circuit 53.

The control circuit 53 controls each part of the tablet terminal 50. The control circuit 53 performs display control for displaying a GUI on the touch panel 52, for example. The control circuit 53 controls each part of the tablet terminal 50 on the basis of input information corresponding to an input operation received by the touch panel 52 and generates transmission information to be transmitted to an external device such as the modality 40 on the basis of the input information. The transmission information includes examination-related information of the modality 40 and the like. The user U can remotely operate the plurality of modalities 40 by performing input operations on the touch panel 52.

The control circuit 53 acquires the usage status of the tablet terminal 50. The usage status of the tablet terminal 50 includes, for example, the modality 40 operated by the tablet terminal 50, details of operation performed on the modality 40 being operated, and the like. Specifically, the usage status of the tablet terminal 50 includes “console operation,” “non-operation,” “imaging screen operation,” “talking with a doctor,” “patient registration,” and the like.

“Console operation” is a state in which the tablet terminal 50 is used to operate the console provided in the modality 40. By operating the tablet terminal 50, the user U can cause the tablet terminal 50 to transmit examination-related information directed to any one of the plurality of modalities 40. “Non-operation” is a state in which the tablet terminal 50 is not operated (not used). “Imaging screen operation” is, for example, a state in which the tablet terminal 50 is used to designate settings and the like of each piece of equipment installed on the pedestal in order to image a subject.

“Talking with a doctor” is a state in which the user U uses the tablet terminal 50 to talk with a doctor. The tablet terminal 50 is provided with a microphone (not shown), and the user U realizes talking with a doctor by causing the microphone to collect his or her vocalized sound and receiving vocalized sound of the doctor from the speaker 56.

“Patient registration” is a state in which the user U uses the tablet terminal 50 to register a new patient (subject) in the HIS 10. The usage status may include other statuses. In response to a request from the medical information processing device 100, the control circuit 53 transmits the usage status of the tablet terminal 50 to the medical information processing device 100 via the network NW using the communication interface 51.

The notification device 54 operates according to control of the control circuit 53. The notification device 54 performs a call notification according to notification information transmitted by the medical information processing device 100. The speaker 56 included in the notification device 54 outputs sound as a call notification. The vibrator 57 vibrates the entire tablet terminal 50 as a call notification. The light emitting device 58 turns on, blinks, or turns off a lamp as a call notification.

The memory 55 stores, for example, a notification method list 59 including notification methods for call notifications. The notification method list 59 is information indicating notification methods for call notifications associated with notification levels included in notification information transmitted by the medical information processing device 100. When the notification method is different, the intensity of notification for calling the user U by the notification device 54 changes. As a notification level increases, the notification device 54 gives stronger stimulation to the user U to notify the user U that the user U is being called.

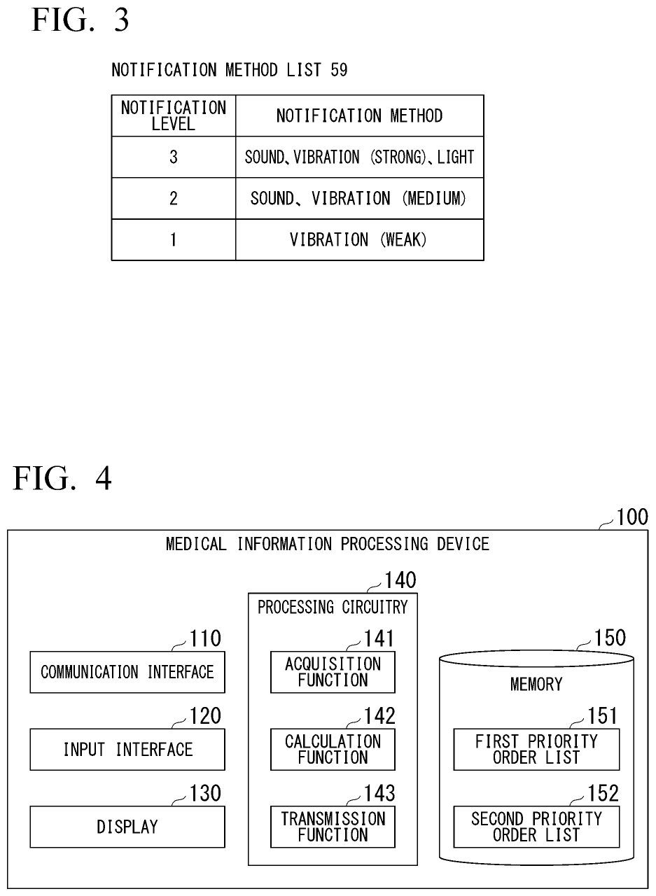

FIG. 3 is a diagram showing an example of the notification method list 59 of the first embodiment. Notification methods included in the notification method list 59 include, for example, notification by the speaker 56 (hereinafter, sound notification), notification by the vibrator 57 (hereinafter, vibration notification), and notification by the light emitting device 58 (hereinafter, optical notification). For example, three levels of “strong,” “medium,” and “weak” are set for vibration notification. In the first embodiment, notification levels are set to three stages, and as the numerical value represented by a notification level increases (a notification priority increases), the notification level increases and stimulation given to the user U increases.

For example, notification level 3 is a notification that provides the highest stimulation to the user U. In the case of notification level 3, all of the sound notification, vibration notification, and optical notification are executed, and the intensity of the vibration notification is set to “strong.” Notification level 2 is a notification that provides medium stimulation to the user U. In the case of notification level 2, the sound notification and the vibration notification are executed, and the vibration notification is set to “medium.” In the case of notification level 1, the notification provides the least stimulation to the user U. At notification level 1, the vibration notification is executed, and “weak” vibration notification is executed.

Although stimulation given to the user U is changed by executing the vibration notification at the intensities of “strong,” “medium,” and “weak,” the sound notification and the optical notification may also change forms to change stimulation given to the user U. For example, in the case of sound notification, stimulation to the user U may be changed by changing the volume or sound quality or by sending a message in response to a notification level. In the case of optical notification, stimulation to the user U may be changed using the amount of light, the color of a light source, blinking, lighting, a blinking speed at the time of blinking, and the like.

FIG. 4 is a block diagram showing an example of a configuration of the medical information processing device 100 of the first embodiment. The medical information processing device 100 includes, for example, a communication interface 110, an input interface 120, a display 130, processing circuitry 140, and a memory 150. The communication interface 110, the input interface 120, and the display 130 in the medical information processing device 100 are provided separately from a communication interface, an input interface, and a display included in the HIS 10, but these may be common.

The communication interface 110 communicates with external devices such as the HIS 10, the RIS 20, the PACS 30, and the modality 40 via a network NW such as a LAN, for example. The communication interface 110 includes, for example, a communication interface such as a NIC.

The input interface 120 receives various input operations from a practitioner and the like, converts the received input operations into electrical signals, and outputs the electrical signals to the processing circuitry 140. The input interface 120 includes, for example, a mouse, a keyboard, a trackball, a switch, a button, a joystick, a touch panel, and the like. The input interface 120 may be, for example, a user interface that receives voice input such as a microphone. In a case in which the input interface 120 is a touch panel, the input interface 120 may also have the display function of the display 130.

Note that in this specification, the input interface is not limited to one that includes physical operation parts such as a mouse and a keyboard. For example, examples of the input interface include electrical signal processing circuitry that receives an electrical signal corresponding to an input operation from external input equipment provided separately from the device and outputs this electrical signal to a control circuit.

The display 130 displays various types of information. For example, the display 130 displays images generated by the processing circuitry 140 and a GUI for receiving various input operations from an operator such as the user U. For example, the display 130 is a liquid crystal display (LCD), a cathode ray tube (CRT) display, an organic electroluminescence (EL) display, or the like.

The processing circuitry 140 includes, for example, an acquisition function 141, a calculation function 142, and a transmission function 143. The processing circuitry 140 realizes these functions by, for example, a hardware processor (computer) executing a program stored in the memory (storage circuit) 150.

The hardware processor means, for example, circuitry such as a CPU, a graphics processing unit (GPU), an application specific integrated circuit (ASIC), or a programmable logic device (for example, a simple programmable logic device (SPLD) or a complex programmable logic device (CPLD)), or a field programmable gate array (FPGA).

Instead of storing the program in the memory 150, the program may be directly incorporated into the circuit of the hardware processor. In this case, the hardware processor realizes the functions by reading and executing the program incorporated into the circuit. The aforementioned program may be stored in advance in the memory 150 or may be stored in a non-transitory storage medium such as a DVD or a CD-ROM and installed in the memory 150 from the non-transitory storage medium when the non- transitory storage medium is set in a drive device (not shown) of the medical information processing device 100.

The hardware processor is not limited to being configured as a single circuit and may be configured as a single hardware processor by combining a plurality of independent circuits to realize each function. Further, a plurality of components may be integrated into one hardware processor to realize each function. Although the hardware processor, the memory, and the like in the medical information processing device 100 are provided separately from the hardware processor, the memory, and the like in the HIS 10, they may be common.

The acquisition function 141 acquires information transmitted by the HIS 10, the RIS 20, the PACS 30, the modality 40, and the tablet terminal 50. The acquisition function 141 acquires, for example, a call request transmitted by the modality 40 and received by the communication interface 110. When the acquisition function 141 has acquired the call request, the acquisition function 141 requests provision of schedules of the user U and the modality 40 from the HIS 10 and requests provision of the usage status of the tablet terminal 50 from the tablet terminal 50.

The acquisition function 141 acquires, for example, the schedule of the user U and the modality 40 transmitted by the HIS 10 and received by the communication interface 110. The acquisition function 141 acquires the usage status of the tablet terminal 50, which is transmitted by the tablet terminal 50 and received by the communication interface 110, for example. The acquisition function 141 checks and acquires details of the work being performed by the user U (hereinafter, details of the work executed by the user U) on the basis of the acquired usage status of the tablet terminal 50. The acquisition function 141 is an example of an acquisition unit. The details of the work executed by the user U is an example of details of user's work.

The calculation function 142 checks details of an examination executed by the user U using the modality 40 (hereinafter, details of an examination by the modality 40) on the basis of the schedules of the user U and the modality 40 acquired by the acquisition function 141. The details of the examination by the modality 40 maybe checked according to the operating state of the modality 40 or the like. In this case, the relationship with the modality 40 that the user U is using may be checked, for example, on the basis of the schedules of the user U and the modality 40.

The details of the examination by the modality 40 also include details such as whether or not the user U is performing the examination using the modality 40 and which modality 40 among the plurality of modalities 40 is being used by the user U. The calculation function 142 calculates a notification priority for each of a plurality of users U on the basis of details of work executed by the users U acquired by the acquisition function 141 and checked details of examinations by modalities 40. For example, the calculation function 142 calculates the notification priority by including details of work being performed by the user when the calculation function 142 calculates the notification priority in the details of user' work. The calculation function 142 is an example of a calculation unit.

The transmission function 143 sets a notification level at the time of issuing a notification to each user U on the basis of the notification priority calculated by the calculation function 142. The transmission function 143 creates notification information including information on the set notification level. The transmission function 143 transmits the created notification information to the tablet terminal 50 managed by each user using the communication interface 110. The transmission function 143 is an example of a transmission unit.

The memory 150 stores, for example, a first priority order list 151 and a second priority order list 152. The first priority order list 151 is information indicating a first priority order associated with details of work performed by the user U. The second priority order list 152 is information indicating a second priority order associated with details of an examination by the modality 40.

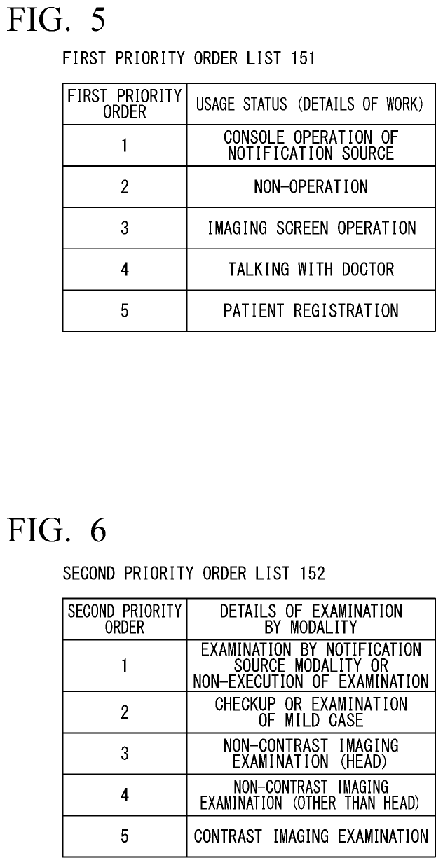

FIG. 5 is a diagram showing an example of the first priority order list 151 of the first embodiment. Priority order “1” is associated with “console operation of notification source.” In addition, priority order “2” is associated with “non-operation,” priority order “3” is associated with “imaging screen operation,” priority order “4” is associated with “talking with a doctor,” and priority order “5” is associated with “patient registration.” In the first embodiment, priority orders of the first priority order list 151 are set to five stages. The smaller the numerical value represented by a priority order decreases, the higher the priority order, and the higher the priority order, the more likely a notification level to increase.

For example, since the user U who is performing “console operation of the notification source” is involved in a notification source modality 40, it is preferable to preferentially notify the user U, and thus the high priority order is set to “1” which is high. In addition, in a case in which information is being transmitted from the tablet terminal 50 to a modality 40 other than the notification source, such as “patient registration,” there is a high possibility that important work is being performed, and thus the priority order is set to “5” which is low. Furthermore, in a case in which the user U is operating the tablet terminal 50, such as “imaging screen operation,” the priority order is set to “3” which is a middle level because there is a high possibility that the importance of the work of the user U is not very high.

FIG. 6 is a diagram showing an example of the second priority order list 152 of the first embodiment. In the second priority order list 152, priority order “1” is associated with a case in which an examination performed by the user U is “an examination performed by a notification source modality 40 or non-execution of an examination.” Further, priority order “2” is associated with “checkup or examination of mild case.” In addition, priority order “3” is associated with “non-contrast imaging examination (head),” priority order “4” is associated with “non-contrast imaging examination (other than head),” and priority order “5” is associated with “contrast imaging examination.” In the first embodiment, priority orders of the second priority order list 152 are set to five stages. The smaller the numerical value represented by a priority order, the higher the priority order, and the higher the priority order, the more likely a notification level to increase.

For example, since it is difficult for the user U who is performing a “contrast imaging examination” to leave the site, the priority order is set to “4” which is low. Note that instead of or in addition to the “contrast imaging examination,” an “examination involving breath-holding” maybe set as an examination with a low priority order (=“4”). In a case in which it is substantially impossible for the user U to leave the place, such as a “contrast imaging examination,” a notification to the user U may not be performed. Furthermore, in the case of “non-contrast imaging examination,” the user U is less likely to leave the site than in the case of a “contrast imaging examination,” and thus the priority order may be set as high as “2” or “3.”

Among “non-contrast imaging examinations,” with respect to a head examination, a period in which the need for the user U to be present during the examination is low continues after an imaging plan has been executed at the beginning of the examination, and thus a situation in which the user U is more likely to leave the site as compared to examinations of parts other than the head may occur. Therefore, the priority order of “non-contrast imaging examination (head)” is set to “2,” and the priority order of “non-contrast imaging examination (other than head)” is set to “3.” Furthermore, there are situations in which it is not highly necessary for the user U to be present depending on attributes of a patient, such as “a patient with a medical checkup or a minor injury.” For this reason, the priority order of a “patient with a medical checkup or minor injury” is set as high as “1.”

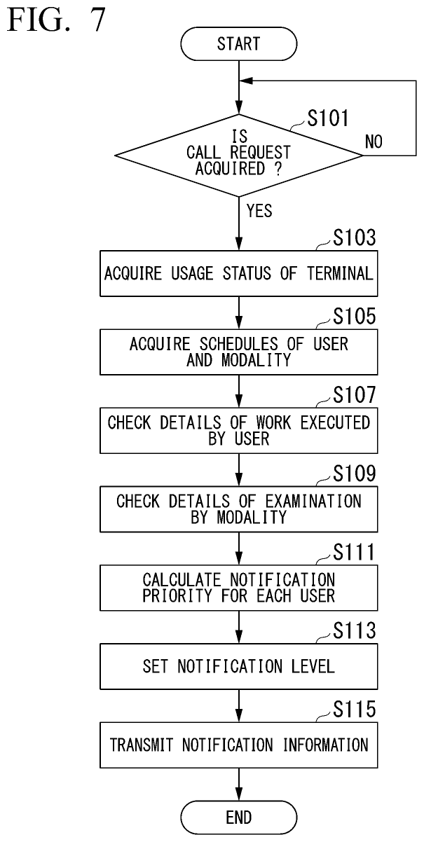

Next, processing in the medical information processing device 100 will be described. FIG. 7 is a flowchart showing an example of processing in the medical information processing device 100 of the first embodiment. In the medical information processing device 100, first, the acquisition function 141 determines whether or not a call request transmitted by any of the plurality of modalities 40 has been acquired (step S101).

If it is determined that the call request has not been acquired, the acquisition function 141 repeats processing of step S101. If it is determined that the call request has been acquired, the acquisition function 141 requests provision of a usage status of a tablet terminal 50 from the tablet terminal 50 and acquires the usage status of the tablet terminal 50 (step S103). Subsequently, the acquisition function 141 requests provision of schedules of a user U and a modality 40 from the HIS 10 and acquires the schedules of the user U and the modality 40 (step S105).

Next, the calculation function 142 checks details of work performed by the user U on the basis of the usage status of the tablet terminal 50 acquired by the acquisition function 141 (step S107). In the first embodiment, the usage status of the tablet terminal 50 directly means the details of the work performed by the user U. Subsequently, the calculation function 142 checks details of an examination by the modality 40 on the basis of the schedules of the user U and the modality 40 acquired by the acquisition function 141 (step S109).

Subsequently, the calculation function 142 calculates a notification priority for each user U on the basis of the checked details of the work performed by the user U and the details of the examination by the modality 40 (step S111). For example, the calculation function 142 sets a first priority order and a second priority order for the details of the work executed by the user U and the details of the examination by the modality 40, respectively, and calculates a notification priority using the set first priority order and second priority order. The calculation function 142 calculates a notification priority through the following procedure, for example.

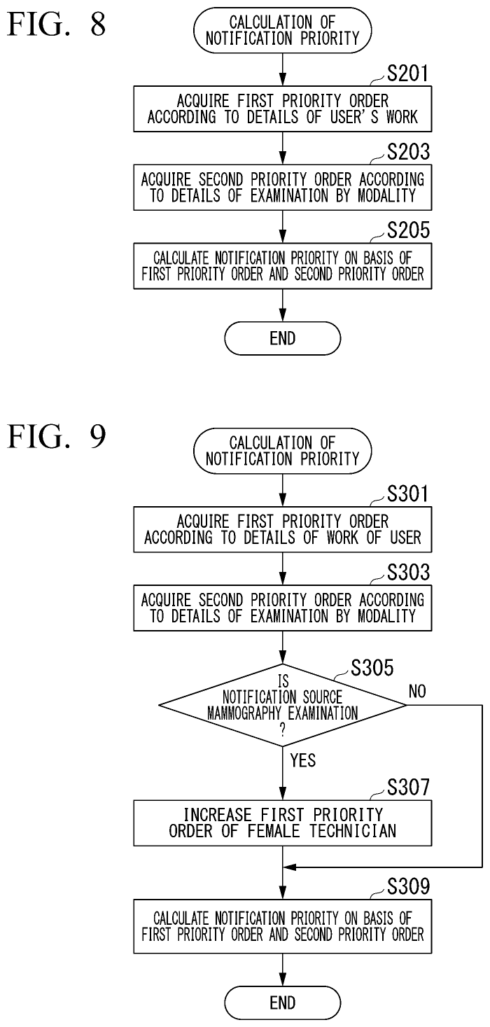

FIG. 8 is a flowchart showing an example of processing in the medical information processing device 100 of the first embodiment. FIG. 8 shows processing of calculating a notification priority. First, the calculation function 142 refers to the first priority order list 151 for the details of the work performed by the user U checked in step S107. The calculation function 142 acquires the first priority order according to the details of the work executed by the user U on the basis of the result of referring to the first priority order list 151 for the details of the work executed by the user U (step S201).

Subsequently, the calculation function 142 refers to the second priority order list 152 for the details of the examination by the modality 40 checked in step S109. The calculation function 142 acquires the second priority order according to the details of the examination by the modality 40 on the basis of the result of referring to the second priority order list 152 for the modality 40 (step S203).

Subsequently, the calculation function 142 calculates a notification priority on the basis of the acquired first priority order list 151 and second priority order list 152 (step S205). The calculation function 142 calculates the notification priority by, for example, adding the numerical value indicated by the first priority order and the rank indicated by the second priority order. In this manner, the medical information processing device 100 ends processing shown in FIG. 8.

The calculation function 142 may calculate the notification priority using other calculation methods. For example, the calculation function 142 may calculate the notification priority by multiplying the first priority order by the second priority order or may calculate the notification priority by adding or multiplying the first priority order and the second priority order multiplied by a predetermined coefficient.

Referring back to the flow shown in FIG. 7, after the calculation function 142 calculates the notification priority for each user U, the transmission function 143 sets a notification level for each of the plurality of users U on the basis of the notification priority calculated by the calculation function 142. The transmission function 143 sets a higher notification level as the notification priority is higher. The transmission function 143 sets the notification level on the basis of results obtained by relatively evaluating the notification priority calculated by the calculation function 142.

For example, in a case in which the notification priority of a first user is “1,” the notification priority of a second user is “4,” and the notification priority of a third user is “6,” the notification level of the first user is set to “3,” the notification level of the second user is set to “2,” and the notification level of the third user is set to “1.” The transmission function 143 may set the notification levels in any way. For example, in a case in which the number of users U and the number of notification levels are the same number, for example “3” and their notification priorities are different, the transmission function 143 sets the notification levels to “3,” “2,” and “1” in descending order of notification priority.

The transmission function 143 may set the notification levels in any way as long as the arrangement of notification levels matches the arrangement of notification priorities of the users U. For example, in a case in which there are two users U with different notification priorities, the transmission function 143 may set notification levels to “3” and “2,” “3” and “1,” or “2” and “1.”

Subsequently, the transmission function 143 generates notification information including information on the set notification levels. The transmission function 143 transmits the generated notification information to the tablet terminals 50 managed by each of the plurality of users U (step S115). Thereafter, the medical information processing device 100 ends processing shown in FIG. 7.

The tablet terminal 50 that has received the notification information transmitted by the transmission function 143 executes notification according to the notification level included in the transmitted notification information. In a case in which the notification level included in the notification information is “3,” the tablet terminal 50 performs notification using sound from the speaker 56, strong vibration from the vibrator 57, and light emission from the light emitting device 58. In a case in which the notification level included in the notification information is “2,” the tablet terminal 50 performs notification using sound from the speaker 56 and moderate vibration from the vibrator 57. In a case in which the notification level included in the notification information is “1,” the tablet terminal 50 performs notification using weak vibration from the vibrator 57.

The medical information processing device 100 of the first embodiment notifies a plurality of users U of a call in a case in which the modality 40 has transmitted a call request for calling a user U. Therefore, when the user U needs to respond to the modality 40 due to an abnormality occurring in the modality 40, the user U can rapidly respond to the modality 40.

Furthermore, at the time of transmitting a call notification to a plurality of users U, the medical information processing device 100 of the first embodiment performs notification in a manner based on a notification level in which a notification level is set in the call notification. Accordingly, a user U who has received the call notification can recognize how much he or she is needed. Therefore, it is possible to enable the user U who is appropriate for the modality 40 to respond.

The tablet terminal 50 maybe provided with an end button for ending a notification for the tablet terminal 50 managed by the user U who operates the tablet terminal 50 or another user U. When a user U who has reached a modality 40 that has output a call request operates the end button, it is possible to curb a situation in which an excessive number of users U gather at the modality 40 that has output the call request.

### (Modified Example of First Embodiment)

Next, a modified example of the first embodiment will be described. the modified example differs from the first embodiment mainly with respect to the procedure of calculating a notification priority. In the modified example, a notification priority is adjusted depending on details of an examination by the modality 40. For example, specifically, in a case in which an examination being performed by the modality 40 is a mammography examination (hereinafter, a mammography examination) in the modified example, the notification priority of a user U, who is a female technician, is adjusted to be high. Alternatively, in a case in which the modality 40 performs an examination of a young person such as a child or an elderly person, the notification priority of a user U who is a technician suitable for the subject of that age may be adjusted to be high.

When the modality 40 transmits a call request in a case in which a mammography examination is being performed, the modality 40 adds the purport to the call request. Furthermore, information on a user U managed by the medical information processing device 100 includes the gender of the user U as an attribute of the user U. The modified example will be described below, focusing on differences from the first embodiment.

FIG. 9 is a flowchart showing an example of processing in the medical information processing device 100 of the modified example. In the modified example, first, the medical information processing device 100 acquires a first priority order according to details of work performed by a user U through the calculation function 142 (step S301). Subsequently, the calculation function 142 acquires a second priority order according to details of an examination by the modality 40 (step S303). Processing up to this point is the same as the first embodiment.

Subsequently, the calculation function 142 refers to a call request transmitted by a notification source modality 40 and determines whether or not the examination being performed by the notification source modality 40 is a mammography examination (step S305). As a result, if it is determined that the examination being performed by the notification source modality 40 is not a mammography examination, the calculation function 142 causes processing to proceed to step S309.

Further, if it is determined that the examination being performed by the notification source modality 40 is a mammography examination, the calculation function 142 increases the first priority order of a user U who is a female technician (step S307). In this case, the calculation function 142 may increase the first priority order of the user

U who is a female technician in any way. For example, the calculation function 142 may increase the first priority order of the user U who is a female technician by one level.

Thereafter, the calculation function 142 calculates a notification priority on the basis of the first priority order list 151 acquired in step S301 and the second priority order list 152 acquired in step S303 or step S305 as in the first embodiment (step S309). In this way, the medical information processing device 100 ends processing shown in FIG. 9.

The medical information processing device 100 of the modified example has the same effects as the first embodiment. Furthermore, in the medical information processing device 100 of the modified example, when the examination being performed by the notification source modality 40 is a mammography examination, the priority order of a female technician is increased. Therefore, it is possible to give priority to female technicians for a mammography examination, and furthermore, it is possible to enable a user U who is appropriate for the modality 40 to respond to the mammography examination.

### (SECOND EMBODIMENT)

Next, a second embodiment will be described. A medical information processing device 100 of the second embodiment differs from the medical information processing device 100 of the first embodiment mainly with respect to processing when notification priorities for a plurality of users are the same, and the device configuration is common in the first and second embodiments. The second embodiment will be described below, focusing on differences from the first embodiment.

The medical information processing device 100 of the second embodiment executes processing according to the flowchart shown in FIG. 7, and differs from the first embodiment with respect to processing of setting a notification level (step S113). Here, processing of the medical information processing device 100 of the second embodiment, particularly processing corresponding to step S113 in FIG. 7, will be described.

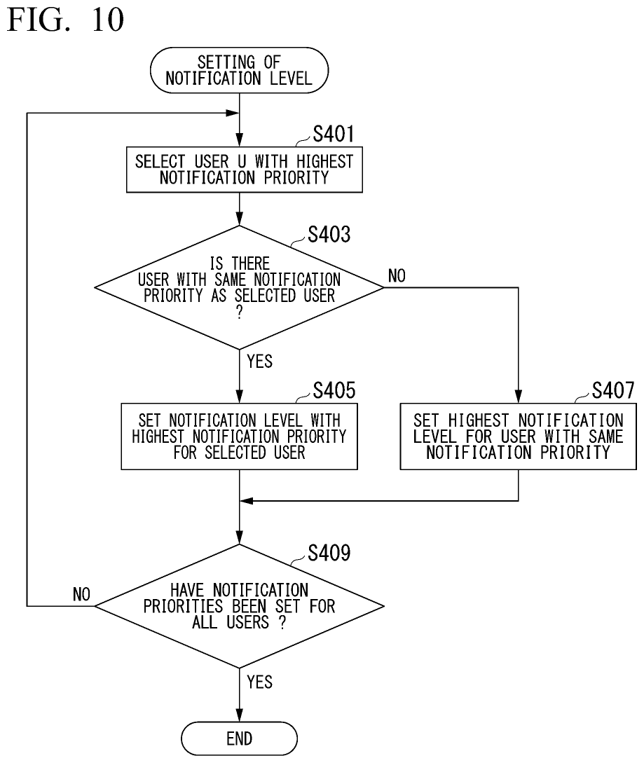

FIG. 10 is a flowchart showing an example of processing in the medical information processing device 100 of the second embodiment. FIG. 10 shows processing corresponding to step S113 in FIG. 7 in the medical information processing device 100 of the second embodiment. Similarly to the first embodiment, the calculation function 142 sets the first priority order and the second priority order for each of a plurality of users U, and then calculates a notification priority for each of the plurality of users U on the basis of the first priority order and the second priority order. The transmission function 143 executes processing shown in FIG. 10 after the calculation function 142 calculates the notification priority.

After the notification priority is calculated by the calculation function 142, the transmission function 143 selects a user U with the highest notification priority from among a plurality of users U for which notification levels have not been set (step S401). Subsequently, the transmission function 143 determines whether or not there is a user U with the same notification priority as the selected user U (step S403).

It is assumed that the transmission function 143 determines that there is a user U with the same notification priority as the selected user U. In this case, the notification priority for the plurality of users U is common. The transmission function 143 sets the highest notification level among a plurality of notification levels to be selected, in this case, notification levels that have not been set, for the user U with the same notification priority as the selected user U (step S405). The highest notification level is the notification level that provides the strongest stimulation to the user.

On the other hand, it is assumed that the transmission function 143 determines that there is no user U with the same notification priority as the selected user U in step S403. In this case, the transmission function 143 sets the highest notification level among the notification levels that have not been set for the selected user U (step S407).

Subsequently, the transmission function 143 determines whether or not notification levels for all users U have been set (step S409). In a case in which the transmission function 143 determines that notification levels for all users U have been set, the medical information processing device 100 ends processing shown in FIG. 10. In a case in which the transmission function 143 determines that notification levels for all users U have not been set, the transmission function 143 returns processing to step S401. In this way, the medical information processing device 100 ends processing shown in FIG. 10.

The medical information processing device 100 of the second embodiment has the same effects as the first embodiment. Furthermore, the medical information processing device 100 of the second embodiment sets the same notification level for users U with the same notification priority. Therefore, it is possible to notify users U with the same notification priority regardless of priority levels. As a result, a user U who is more appropriate for the modality 40 can respond.

### (THIRD EMBODIMENT)

Next, a third embodiment will be described. A medical information processing device 100 of the third embodiment differs from the medical information processing device 100 of the first embodiment mainly in that it is built into the modality 40 whereas the medical information processing device 100 of the first embodiment is provided independently from the modality 40. The third embodiment will be described below, focusing on differences from the first embodiment.

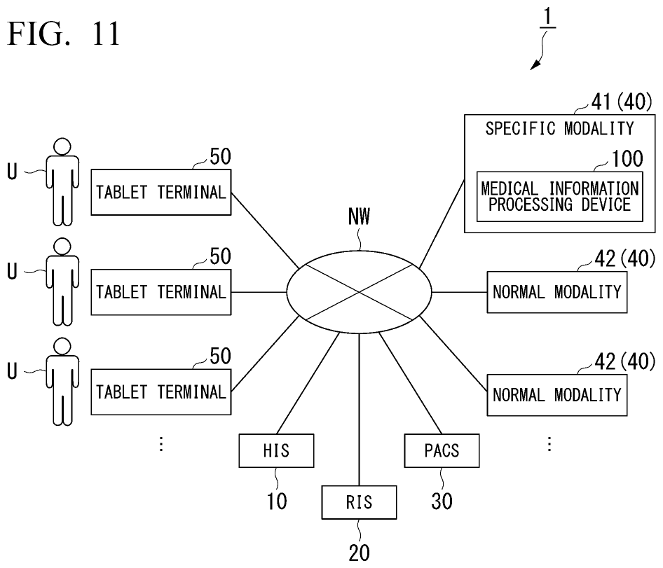

FIG. 11 is a block diagram showing an example of a configuration of an in- hospital system 1 of the third embodiment. The in-hospital system 1 of the third embodiment includes, as modalities 40, a specific modality 41 having the medical information processing device 100 built therein and a normal modality 42 that does not include the medical information processing device 100. The medical information processing device 100 is provided, for example, in a console in the specific modality 41.

The in-hospital system 1 of the third embodiment includes one specific modality 41 and a plurality of normal modalities 42. The specific modality 41 has substantially the same configuration as the normal modality 42 except that it includes the medical information processing device 100. The normal modality 42 has the same configuration as the modality 40 in the first embodiment.

The medical information processing device 100 in the in-hospital system 1 of the third embodiment has the same effects as the first embodiment. Furthermore, the medical information processing device 100 of the third embodiment is built into the modality 40 (specific modality 41). Therefore, the space for installing the independent medical information processing device 100 can be reduced. In the third embodiment, the in-hospital system 1 includes one specific modality 41 and a plurality of normal modalities 42, but the numbers of specific modalities 41 and normal modalities 42 may be changed as appropriate. For example, the in-hospital system 1 mayinclude a plurality of specific modalities 41 or may include a single normal modality 42.

### (FOURTH EMBODIMENT)

Next, a fourth embodiment will be described. A medical information processing device 100 of the fourth embodiment differs from the medical information processing device 100 of the first embodiment mainly with respect to the manner of checking details of work performed by a user, and the device configuration is common in the first and fourth embodiments. The fourth embodiment will be described below, focusing on differences from the first embodiment.

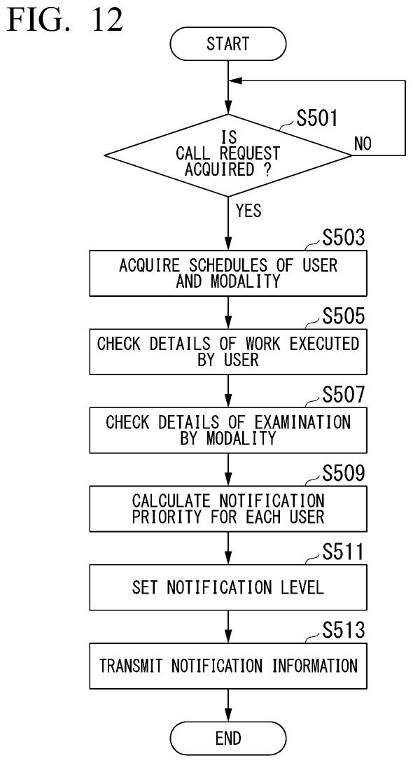

FIG. 12 is a flowchart showing an example of processing in the medical information processing device 100 of the fourth embodiment. Similarly to the first embodiment, first, the medical information processing device 100 of the fourth embodiment determines whether or not a call request has been acquired in the acquisition function 141 (step S501) and repeats processing of step S501 until a call request is acquired.

Subsequently, the acquisition function 141 acquires the usage status of the tablet terminal 50 in the first embodiment. In contrast, the acquisition function 141 requests provision of schedules of users U and modalities 40 from the HIS 10 without acquiring the usage status of the tablet terminal 50 and acquires the schedules of the users U and the modalities 40 in the fourth embodiment (step S503).

Subsequently, the calculation function 142 checks details of work performed by the users U on the basis of the schedules of the users U and the modalities 40 acquired by the acquisition function 141 instead of the usage status of the tablet terminal 50 (step

S505). The calculation function 142 checks the details of the work performed by the users U by referring to the current time for the schedules of the users U and the modalities 40, for example.

Thereafter, similarly to the first embodiment, the calculation function 142 checks details of examinations by the modalities 40 (step S507), and then calculates the notification priority for each user (step S509). Further, the transmission function 143 sets a notification level on the basis of the notification priority (step S511) and transmits notification information including the set level to the tablet terminal 50 (step S513). In this way, the medical information processing device 100 of the fourth embodiment ends processing shown in FIG. 12.

The medical information processing device 100 of the fourth embodiment has the same effects as the first embodiment. Furthermore, the medical information processing device 100 of the fourth embodiment checks details of work performed by the users U on the basis of schedules of the users U and the modalities 40. Therefore, it is possible to omit processing of requesting provision of the usage status from the tablet terminal 50 or receiving the usage status of the tablet terminal 50 transmitted from the tablet terminal 50. Accordingly, compared to the first embodiment, the device and processing can be simplified.

In the fourth embodiment, the medical information processing device 100 checks details of current work executed by the users U on the basis of the schedules of the users U and the modalities 40. On the other hand, the medical information processing device 100 may check details of work to be executed by the users U in the future instead of or in addition to the details of the current work executed by the users U. In this case, the calculation function 142 may calculate notification priorities by including details of work scheduled to be performed by the users in the future in the details of user's work.

Although it is difficult to check the details of work to be executed by the users U in the future only using the usage status of the tablet terminal, the details of work to be executed by the users U in the future can be estimated (checked) on the basis of the schedules of the users U and the modalities 40. It is possible to set notification priorities of the plurality of users U more appropriately by calculating the notification priorities by including the details of work scheduled to be performed by the users U in the future in the details of user's work.

Although the user U is a technician in each of the above embodiments, the user U may be someone other than a technician. The user U may be a doctor or a nurse, for example. Further, although a call request is transmitted by the modality 40 in the above embodiments, the call request may be transmitted by a device other than the modality 40. The call request may be transmitted by the tablet terminal 50 of the user U who is dealing with the modality 40, for example, when the user who is dealing with the modality 40 requires more personnel.

Although the call request is transmitted in a case in which a treatment by the user U is required during an examination in each of the above embodiments, the call request may be transmitted in cases other than a case in which a treatment by the user U is required during an examination. The call request may be transmitted, for example, in a case in which the end of an examination of a subject is approaching and thus a response of the user U is required due to the end of the examination.

According to at least one embodiment described above, it is possible to enable an appropriate technician or the like to respond by including an acquisition unit that acquires a call request for calling a user to a medical image diagnostic device, a calculation unit that calculates a notification priority for each of a plurality of users on the basis of at least one of usage statuses of a plurality of terminals managed by the plurality of users and details of user's work, and a transmission unit that transmits notification information for causing the terminals to output a call notification according to the notification priority to the plurality of terminals.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device comprising processing circuitry configured to:
acquire a call request for calling a user to a medical image diagnostic device;
calculate a notification priority for each of a plurality of users on the basis of at least one of usage statuses of a plurality of terminals managed by a plurality of users or details of work of the plurality of users; and
transmit notification information for causing the plurality of terminals to output a call notification according to the notification priority to the plurality of terminals.

2. The medical information processing device according to claim 1, wherein the processing circuitry is configured to calculate the notification priority on the basis of the details of work of the plurality of users including details of work being performed by the users at the time of calculating the notification priority.

3. The medical information processing device according to claim 1, wherein the processing circuitry is configured to calculate the notification priority on the basis of the details of work of the plurality of users including details of work scheduled to be performed by the users in the future.

4. The medical information processing device according to claim 1, wherein the processing circuitry is configured to cause the plurality of terminals to output a call notification for increasing stimulation given to the plurality of users as the notification priority increases.

5. The medical information processing device according to claim 1, wherein, in a case in which the notification priority for the plurality of users is common, the processing circuitry is configured to cause the plurality of terminals to output a call notification of a notification level providing a strongest stimulation to the plurality of users from among a plurality of notification levels to be selected.

6. The medical information processing device according to claim 1, wherein the call notification includes at least one of sound, vibration, or light emission output by the plurality of terminals.

7. The medical information processing device according to claim 1, wherein the processing circuitry is configured to adjust the notification priority according to details of an examination to be performed by the medical image diagnostic device.

8. The medical information processing device according to claim 1, wherein the medical information processing device is built into the medical image diagnostic device.

9. A medical information processing method, using a computer, comprising:
acquiring a call request from a medical image diagnostic device;
calculating a notification priority for each of a plurality of users on the basis of at least one of usage statuses of a plurality of terminals managed by the plurality of users or details of work of the plurality of users; and
transmitting notification information for causing the plurality of terminals to output a call notification according to the notification priority to the plurality of terminals.

10. A computer-readable non-transitory storage medium storing a program causing a computer to:
acquire a call request from a medical image diagnostic device;
calculate a notification priority for each of a plurality of users on the basis of at least one of usage statuses of a plurality of terminals managed by the plurality of users or details of works of the plurality of users; and
transmit notification information for causing the plurality of terminals to output a call notification according to the notification priority to the plurality of terminals.
